(19) United States
(12) Patent Application Publication
(10) Pub. No.: US 2004/0225258 A1
(43) Pub. Date: Nov. 11, 2004

(54) Tamper evident overcap for a container

(76) Inventor: Ernest Balestracci, Iselin, NJ (US)

Correspondence Address: BRACCO RESEARCH USA INC., 305- COLLEGE ROAD EAST, PRINCETON, NJ, 08540, US

(21) Appl. No.: 10/867,030

(22) Filed: Jun. 14, 2004

Related U.S. Application Data
(63) Continuation-in-part of application No. 10/109,827, filed on Mar. 30, 2002.

(51) Int. Cl. A61M 5/00

(52) U.S. Cl. 604/111; 604/187

(57) ABSTRACT

A tamper evident protective cap for a prefilled syringe having a pharmaceutical or biological fluid is provided. The barrel of the container is equipped with a ring. The protective cap consisting of top and bottom portions are connected by a frangible area. The bottom portion contains a flange to engage the ring. Once the flange is engaged to the ring, the engagement prevents the removal of the tamper evident cap unless the top portion is intentionally separated from the bottom portion at the frangible area. If the top portion is intentionally separated, the bottom portion will remain engaged to the ring and the disengaged top portion will indicate tamper evidence.

A method of removing a tamper evident cap from a pre-filled syringe is also provided.

### RELATED APPLICATION

This application is a continuation-in-part of U.S. patent application Ser. No. 10/109,827 filed Mar. 30, 2002, which is incorporated by reference herein in its entirety.

### FIELD OF THE INVENTION

The present invention relates to a tamper evident protector overcap for a prefilled syringe barrel. More particularly, the invention relates to a cap for a syringe barrel containing a liquid medication therein for securely holding a closure in the tapered tip the syringe barrel and serving as a tamper evident indicator.

### BACKGROUND OF THE INVENTION

Prefilled syringe barrels or cartridges containing injectable solutions therein are stoppered by elastomeric closures, such as soft rubber stoppers at the distal, tapered end thereof, while the proximal end of the barrels are closed by slidable plungers. The prefilled syringe barrels or cartridge are sterilized, such as by autoclaving, and packaged ready for use.

It has been observed that during in-line processing, handling, and sterilizing of the prefilled barrels, some polymeric or elastomeric closures were missing from the tips of the barrels resulting in rejects. Also, during shipment of the finished product and handling by healthcare professionals some untipped barrels were observed which necessitated discarding of batches containing failed samples. For product integrity a corrective measure was indicated to prevent the polymeric or elastomeric closure from becoming dislodged from the tip of the barrel.

More importantly, it has also been recognized that untipped barrels, whether the damage occurred during shipment or handling, attracts the suspicion that the product was tampered with. Such possible tampering is a concern for both the National Regulating Authorities and the manufacturers who are required to insure safety, efficacy and the product integrity.

The prior art has provided various tamper evident closures for syringes.

For example, tamper evident syringes may be characterized in that the syringe barrel, the cap, and the plunger rod are covered with a tubular sealing device that is made from a heat-shrinkable film and which has been shrunk under heat so that it adheres closely to the surface of those members. The sealing device comprises a tube and a tear tape. The tube is formed of a transparent heat-shrinkable film. The tear tape is attached by bonding to the inner surface of the tube from one end to the other in the longitudinal direction.

Another example is a hypodermic syringe used with a needle for lyophilized medicament comprising: a syringe body having a piston therein equipped with a tip cap at its distal end; an elastomeric plug having a passage channel closing the neck portion of the syringe body. The protector cap and tip cap are integral with each other and can be moved axially to open and close the syringe. The protector cap consists of a top portion and a bottom portion, the two parts being held together by a weakened portion. The center of the protector cap is provided with a small hole through which the tip cap can be viewed. In use, the top portion of the protector cap is snapped off at the weakened portion, and the tip cap is taken off and discarded. A needle is then fitted in the passage channel of the elastomeric plug to access the content of the syringe.

Still another example is a syringe cap assembly placed on the distal end of a syringe. The assembly includes: an elastomeric insert having a passage therein; a retaining collar which fits over the elastomeric insert to hold the insert in place; a plug or tip cap is engaged in the insert to block the passage in the insert; and a retaining safety cap fitted over the tip cap. The end wall of the retaining safety cap is formed with a hole in its center and is slightly smaller in diameter than the plug so that the user can ascertain that the plug is properly in its place without opening the assembly.

In use the safety cap is pulled, twisted, and lifted off the assembly. The plug is then lifted off to expose the collar, and a needle assembly is fitted to the collar.

A further example is a prefilled syringe with break-away tip seal which closes the passageway to the content of the syringe. A score means is provided adjacent to the tip for accommodating removal of the sealed tip.

An object of the present invention is to provide a prefilled tamper evident syringe or cartridge barrel which makes apparent the unauthorized use of the medical fluid contained in the barrel of the syringe or cartridge or at least warns healthcare professionals that such unauthorized use may have occurred.

Another object of the present invention is to provide tamper evident syringe or cartridge barrels the content of which is easily accessed by the healthcare professionals while their unauthorized use is readily apparent.

A further object of the present invention is to provide a tamper evident syringe or cartridge barrel the content of which can be accessed by luer connections or a tubing conduit so as to avoid the use of “sharps” and thereby preventing needle stick injuries.

### SUMMARY OF THE INVENTION

In accordance with the present invention, an overcap is provided for a syringe or cartridge barrel containing a pharmaceutical or biological liquid. The overcap is designed to indicate unauthorized use of the content of the syringe or cartridge barrel.

Separation of the top portion of the overcap from the bottom portion thereof prior to use indicates evidence to the healthcare professional that the product may have been tampered with and should not be used.

In one embodiment of the present invention, a combination of a tamper evident protective cap and a pre-filled syringe is provided. The combination includes a syringe barrel of cylindrical configuration having a longitudinal axis that extends between a distal end and a proximal end.

The syringe barrel includes an inner surface that defines a cylindrical chamber having a distal end and a tapered tip that terminates at the distal end of the syringe barrel. The tip has a bore therethrough and is equipped with a Luer lock collar. The bore is stoppered by an elastomeric closure. The syringe barrel further includes a ring that is disposed at the distal end of the cylindrical chamber.

The combination also includes a tamper evident cap that is removably engaged with the distal end of the syringe barrel. The tamper evident cap provides a cylindrical top portion that terminates in a flat circular surface that conforms to the elastomeric closure that is disposed in the tapered tip of the syringe barrel.

The tamper evident cap further includes a cylindrical bottom portion that is connected to the cylindrical top portion by a frangible area. The frangible area allows the cylindrical top portion to be removed from the cylindrical bottom portion. The cylindrical bottom portion terminates in an open end and has a flange that extends inwardly that is designed to engage the ring such that the engagement prevents removal of the tamper evident protective cap unless the top portion is intentionally separated from the bottom portion at the frangible area. If the top portion is intentionally separated, the bottom portion remains engaged to the ring and the disengaged top portion indicates tamper evidence.

The syringe barrel is made from a material selected from the group consisting of polyolefin polymers, polyolefin copolymers, polypropylene, olefin polymers, olefin copolymers, cyclic olefins, polyester or methylpentene.

The tamper evident protective cap is made from a polymer material selected from the group consisting of polyethylene, polypropylene, polystyrene, polycarbonate, polymethylpentene, cyclic olefin co-polymers, acrylic polymers and methacrylic polymers. The elastomeric closure is made from a material selected from the group consisting of natural rubber, butyl or halobutyl rubber. The elastomeric closure is further made from a plastic, an elastomeric compound or a combination of plastic and elastomeric compound.

In another embodiment, the cylindrical top portion of the tamper evident protective cap further provides at least one air vent.

In another embodiment, the cylindrical top portion of the tamper evident protective cap further provides a plurality of ribs.

In another embodiment, the cylindrical top portion of the tamper evident protective cap further provides three air vents.

In another embodiment, the cylindrical top portion of the tamper evident protective cap further provides twelve ribs.

In another embodiment, the cylindrical top portion of the tamper evident protective cap has a first diameter and the cylindrical bottom portion has a second diameter and wherein the first diameter is larger than the second diameter.

In another embodiment, the cylindrical top portion of the tamper evident protective cap has a first diameter and the cylindrical bottom portion has a second diameter, wherein the first diameter is about 0.3 mm to 1.3 mm larger than the second diameter.

In another embodiment, the cylindrical top portion of the tamper evident protective cap has a first diameter and the cylindrical bottom portion has a second diameter, wherein the first diameter is about 0.8 mm larger than the second diameter.

In another embodiment, the syringe barrel includes a means of releasably engaging an injection device.

In another embodiment, the syringe barrel includes a locking mechanism that is disposed on the syringe barrel for interfacing with an injection device.

In another embodiment, the syringe barrel includes at least one locking tab that is disposed at the proximal end of the syringe barrel for interfacing with an injection device. The at least one locking tab may further provide a first pair of locking tabs that are positioned 180° opposite a second pair of locking tabs. Both pairs of locking tabs are disposed on an outer surface of the syringe barrel and about the longitudinal axis.

The at least one locking tab may also provide a third locking tab that is positioned 180° opposite a fourth locking tab. The third and fourth locking tabs are disposed on the outer surface of the syringe barrel and about the longitudinal axis. In this embodiment, the first and second pairs of locking tabs are provided 90° offset from the third and fourth locking tabs. This embodiment may further include a flat ring that extends away from the longitudinal axis. The flat ring is disposed at the proximal end of the syringe barrel.

The present invention also provides a method of removing a tamper evident cap from a pre-filled syringe. The method includes the steps of a) providing a tamper evident cap that includes 1) a cylindrical top portion and 2) a cylindrical bottom portion that is connected to the cylindrical top portion at a frangible seal and b) holding the cylindrical bottom portion with a first hand and c) grasping and twistably separating the cylindrical top portion with a second hand, the top portion separating from the bottom portion at the frangible seal.

In another embodiment, the present invention provides a further combination of a tamper evident protective cap and a pre-filled syringe. The combination includes a syringe barrel of cylindrical configuration having a longitudinal axis extending between a distal end and a proximal end.

The syringe barrel includes an inner surface that defines a cylindrical chamber having a distal end and a tapered tip that terminates at the distal end. The tip having a bore therethrough and is equipped with a Luer lock collar. The bore is stoppered by an elastomeric closure. The syringe barrel also includes at least two tabs that project outward that are disposed at the distal end of the cylindrical chamber.

The combination further includes a tamper evident cap that is removably engaged with the tapered distal end of the syringe barrel. The tamper evident cap has a cylindrical top portion that terminates in a flat circular surface that conforms to the elastomeric closure that is disposed in the tapered tip of the syringe barrel

The tamper evident cap also has a cylindrical bottom portion that is connected to the cylindrical top portion by a frangible area. The frangible area allows the cylindrical top portion to be removed from the cylindrical bottom portion. The cylindrical bottom portion terminates in an open end and has a flange that extends inwardly that is designed to engage the at least two tabs such that the engagement prevents the removal of the tamper evident protective cap unless the top portion is intentionally separated from the bottom portion at the frangible area. If the top portion is intentionally separated, the bottom portion remains engaged to the at least two tabs and the disengaged top portion indicates tamper evidence.

In another embodiment, the present invention provides a fuirther combination of a tamper evident protective cap and a pre-filled syringe. The combination includes a syringe barrel of cylindrical configuration having a longitudinal axis extending between a distal end and a proximal end.

The syringe barrel includes an inner surface that defines a cylindrical chamber having a distal end and a tapered tip that terminates at the distal end. The tip having a bore therethrough and is equipped with a Luer lock collar. The bore is stoppered by an elastomeric closure. The barrel also includes a shoulder that is disposed at the distal end of the cylindrical chamber. The shoulder has an annular groove that is disposed on an outer surface.

The combination further includes a tamper evident cap that is removably engaged with the distal end of the syringe barrel. The tamper evident cap has a cylindrical top portion that terminates in a flat circular surface that conforms to the elastomeric closure that is disposed in the tapered tip of the syringe barrel.

The tamper evident cap also includes a cylindrical bottom portion that is connected to the cylindrical top portion by a frangible area. The frangible area allows the cylindrical top portion to be removed from the cylindrical bottom portion. The cylindrical bottom portion terminates in an open end and has a flange that extends inwardly that is designed to engage the groove such that the engagement prevents the removal of the tamper evident protective cap unless the top portion is intentionally separated from the bottom portion at the frangible area. If the top portion is intentionally separated, the bottom portion remains engaged to the annular groove and the disengaged top portion indicates tamper evidence.

In another embodiment, the annular groove is v-shaped.

In another embodiment, the annular groove is semi-circular in shape.

In another embodiment, the flange is biased away from the distal end.

In another embodiment, the flange is biased toward the distal end.

In another embodiment, the present invention provides a combination of a tamper evident protective cap and a pre-filled syringe. The combination includes a syringe barrel of cylindrical configuration having a longitudinal axis that extends between a distal end and a proximal end.

The syringe barrel includes an inner surface that defines a cylindrical chamber having a distal end and a tapered tip that terminates at the distal end of the syringe barrel. The tip has a bore therethrough and is equipped with a Luer lock collar. The bore is stoppered by an elastomeric closure. The syringe barrel further includes a ring that is disposed at the distal end of the cylindrical chamber.

The combination further includes a tamper evident cap that is removably engaged with the tapered distal end of the syringe barrel. The tamper evident cap has a cylindrical top portion that terminates in a flat circular surface that conforms to the elastomeric closure that is disposed in the tapered tip of the syringe barrel.

The tamper evident cap also includes a cylindrical bottom portion that is connected to the cylindrical top portion by a frangible area. The frangible area allows the top portion to be removed from the bottom portion. The bottom portion terminates in an open end and includes at least two wings that project away from the longitudinal axis and a flange that extends inwardly that is designed to engage the ring such that the engagement prevents the removal of the tamper evident protective cap unless the top portion is intentionally separated from the bottom portion at the frangible area. If the top portion is intentionally separated, the bottom portion remains engaged to the ring and the disengaged top portion indicates tamper evidence.

### BRIEF DESCRIPTION OF THE DRAWINGS

FIG. 1 is a perspective view of a syringe or cartridge barrel equipped with an overcap and containing a pharmaceutical or a biological liquid therein;

FIG. 2 is a perspective view of a syringe or cartridge barrel without an overcap and containing a pharmaceutical or a biological liquid therein;

FIG. 3 is a perspective view of an overcap constituting one embodiment of the present invention;

FIG. 4 is a top plan view of the overcap shown in FIG. 3;

FIG. 5 is a bottom plan view of the overcap shown in FIG. 3;

FIG. 6 is a side-elevational view of the overcap shown in FIG. 3;

FIG. 7 is a perspective view of an overcap constituting another embodiment of the present invention;

FIG. 8 is a top plan view of the overcap shown in FIG. 7;

FIG. 9 is a bottom plan view of the overcap shown in FIG. 7;

FIG. 10 is a side-elevational view of the overcap shown in FIG. 7;

FIG. 11 is a side-elevational view of a combination tamper evident protective cap and prefilled syringe constituting another embodiment of the present invention;

FIG. 12 is a cross-sectional view of the combination tamper evident protective cap and prefilled syringe taken along line 12-12 of FIG. 11;

FIG. 13 is a perspective view of another embodiment of a prefilled syringe without an overcap and containing a pharmaceutical or a biological liquid therein of the present invention;

FIG. 14 is a side-elevational view of a combination tamper evident protective cap and prefilled syringe combination constituting another embodiment of the present invention;

FIG. 15 is a cross-sectional view of the combination tamper evident protective cap and prefilled syringe taken along line 15-15 of FIG. 14;

FIG. 16 is a side-elevational view of a tamper evident protective cap and prefilled syringe combination constituting another embodiment of the present invention;

FIG. 17 is a cross-sectional view of the combination tamper evident protective cap and prefilled syringe taken along line 17-17 of FIG. 16;

FIG. 18 is a side-elevational view of a tamper evident protective cap and prefilled syringe combination constituting another embodiment of the present invention; a and

FIG. 19 is a cross-sectional view of the combination tamper evident protective cap and prefilled syringe taken along line 19-19 of FIG. 18;

### DETAILED DESCRIPTION OF THE INVENTION

While the invention includes embodiments in many different forms, there is shown in the drawings and will herein be described in detail preferred embodiments of the invention with the understanding that the present disclosure is to be considered as exemplary of the principles of the invention and is not intended to limit the invention to the embodiments illustrated.

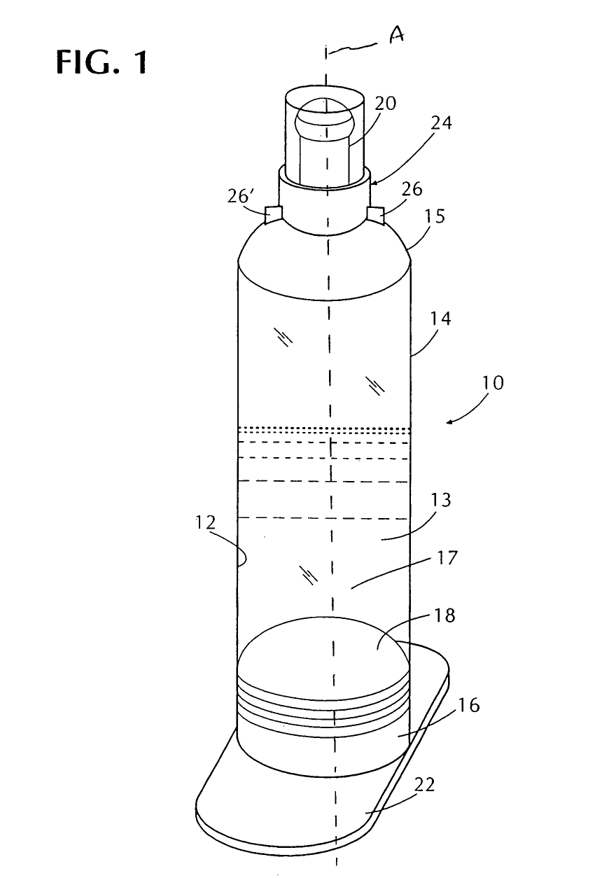

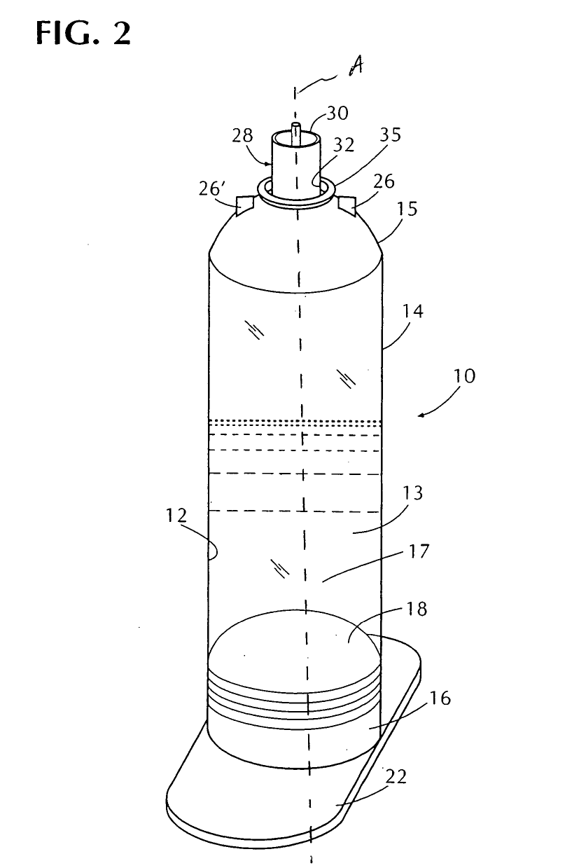

FIGS. 1 and 2 show a syringe or cartridge barrel (hereinafter referred to as a barrel) in perspective views generally designated by the numeral 10 and an overcap generally designated by the numeral 24. FIG. 1 shows the barrel with the overcap, while FIG. 2 shows the barrel without the overcap.

The syringe or cartridge barrel, made of glass or a polymeric material, has an inner surface 12 defining a cylindrical chamber 13 for retaining a pharmaceutical or biological liquid 17 therein, such as a liquid x-ray contrast medium. The barrel has a distal end 14 terminating in a tapered tip 15 having a bore therethrough to which an injection needle or a luer connector with a tubing conduit can be attached, and a proximal end 16 for receiving a plunger 18 which retains the pharmaceutical or biological liquid in the barrel and which, upon use, expels the pharmaceutical or biological liquid from the barrel when an external pressure is exerted on the plunger. A longitudinal axis “A” extends between the distal and proximal ends of the barrel.

For purposes of the description of the prior art and present invention, the term “distal end” refers to the end furthest from the end which receives the piston, whereas the term “proximal end” refers to the end which receives the piston. Further, the term “inward ” refers to a direction moving towards the longitudinal axis, whereas the term “outward” refers to a direction moving away from the longitudinal axis.

The tapered tip 15 having a bore therein is stoppered by a resilient closure 20, such as an elastomeric closure or a soft rubber stopper, for hermetically sealing the distal end of the barrel. At its proximal end 16 the barrel is equipped with an integral flange 22 to facilitate the handling of the barrel. When the pharmaceutical or biological liquid is an injectable solution, the barrel along with its content is sterilized, preferably by autoclave. After sterilization the barrel is packaged and stored ready for use when needed. Delivery of the injectable solution is accomplished by removing the overcap from the distal end of the barrel, and from the tapered tip of the barrel and attaching an injection needle or luer connector onto the tapered tip of the barrel.

The barrel is equipped with two opposing protuberances or knobs 26 and 26′ jutting out from the distal end 14 of the barrel adjacent to the tapered tip 15. The function of these protuberances or knobs will be pointed out as the description of the invention proceeds.

The tapered tip 15 of the barrel terminates in a typical female luer connector 28, generally designated, for attachment of a male luer connector or an injection needle thereto. The luer connector is integral with the tapered tip of the barrel and comprises an open distal end 30, and a closed proximal end 32. At the proximal end of the female luer connector and spaced from protuberance or knobs 26 and 26′, there is provided a ring 35 surrounding the female luer connector 28. The outside diameter of the ring is slightly smaller than the inside diameter of the overcap thereby allowing turning of the overcap either in clockwise or counter-clockwise direction.

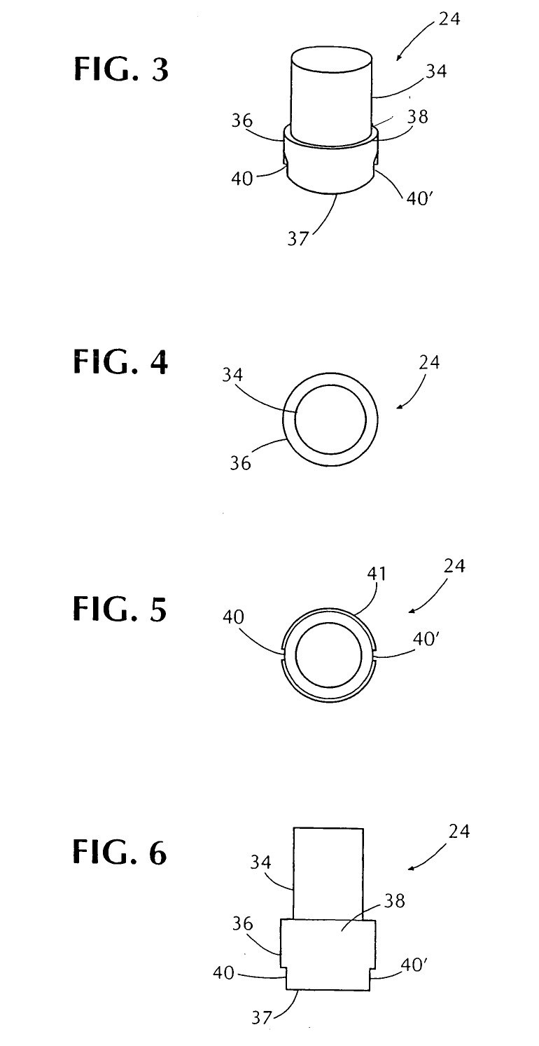

FIGS. 3, 4, 5, and 6 shows the overcap, generally designated at 24, in various views.

FIG. 3 is a perspective view of the overcap comprising: a top portion 34, and a bottom portion 36 separated by a frangible portion 38. The bottom portion has a slightly larger diameter than the top portion. The proximal end 37 of the bottom portion is provided with two cut-outs or notches 40 and 40′ on opposite sides of the bottom portion. The notches are designed to engage protuberances or knobs 26 and 26′ at the distal end of the barrel.

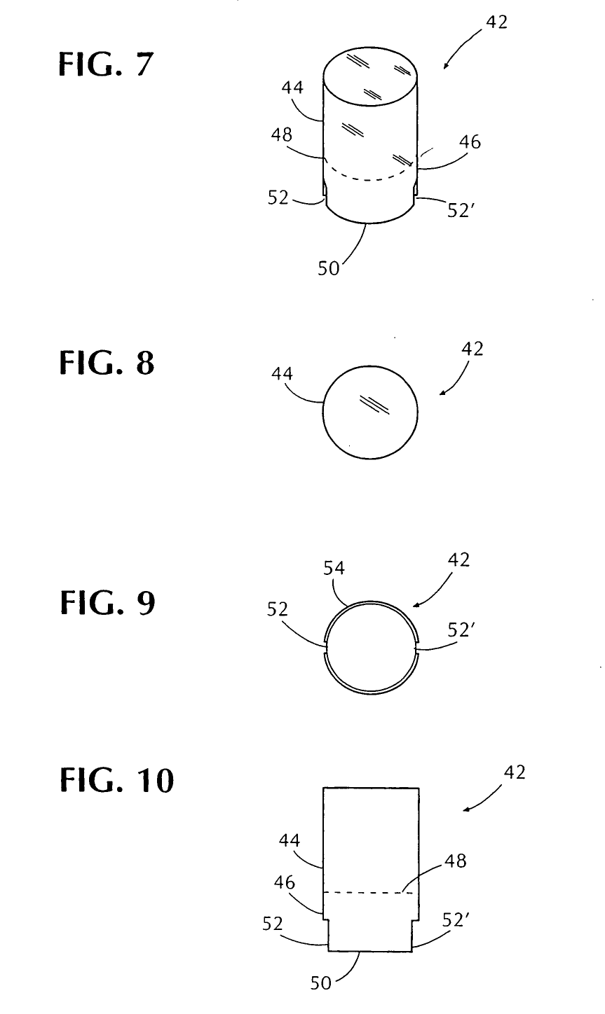

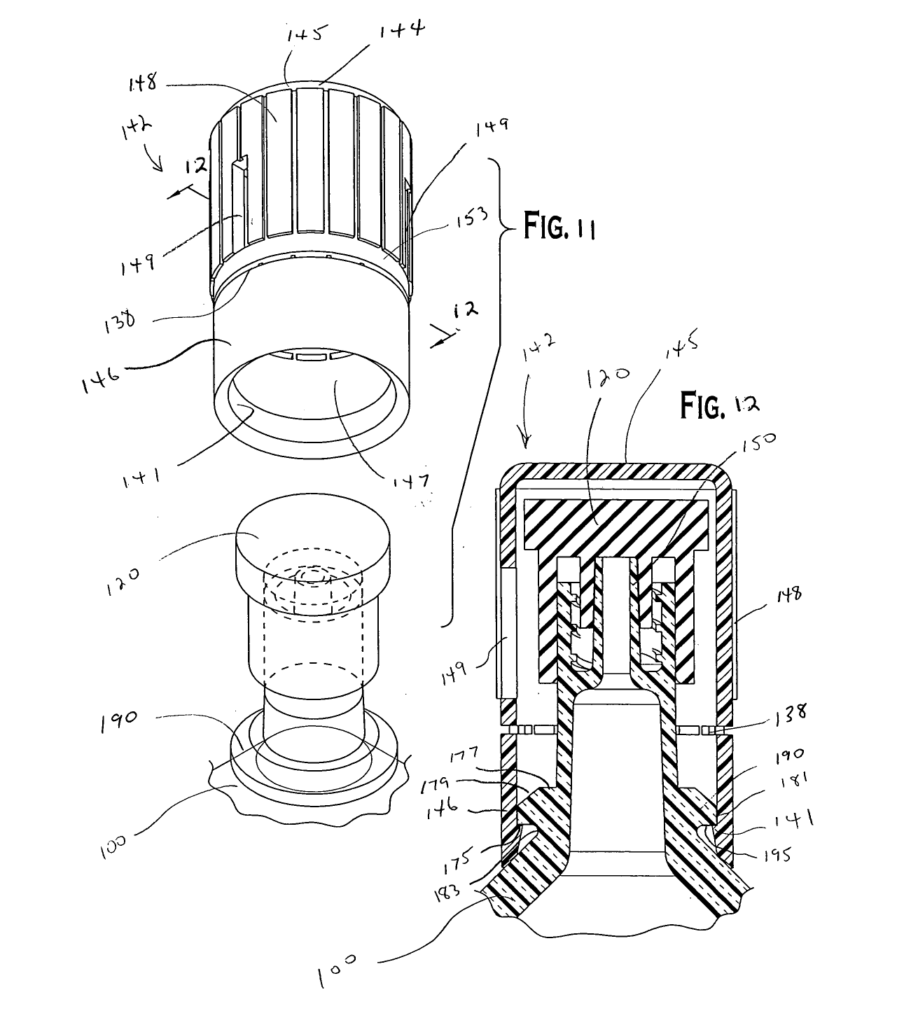

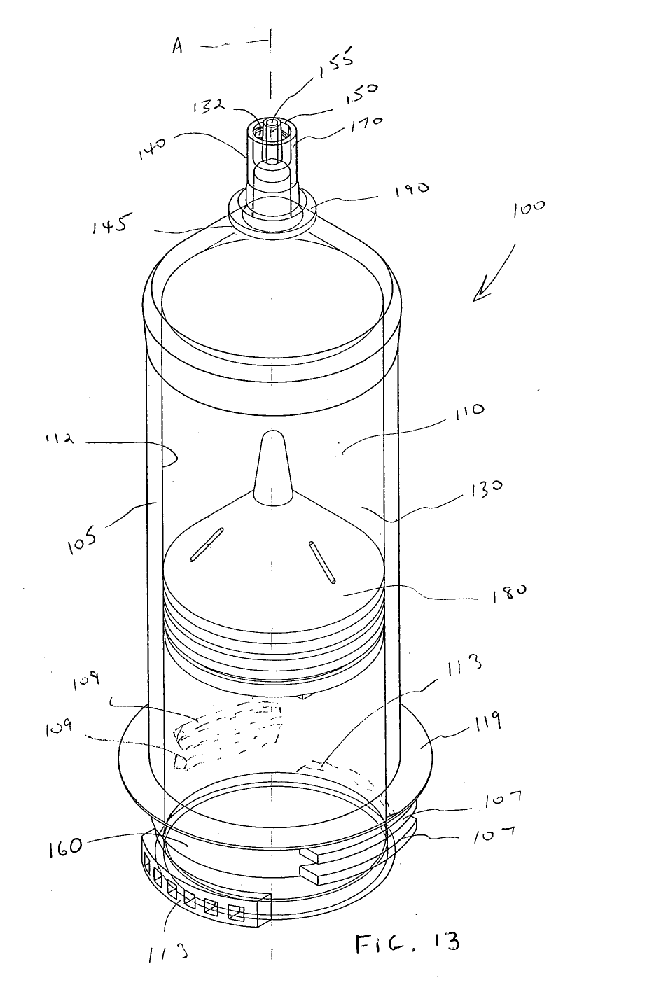

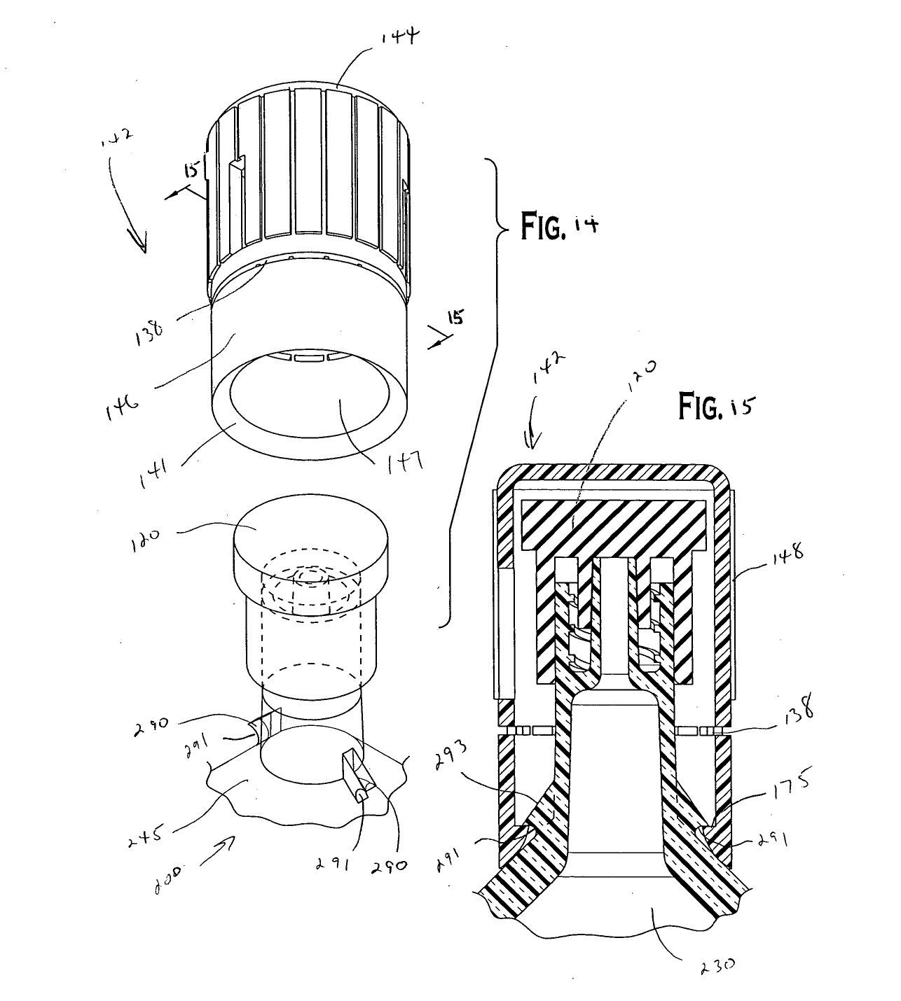

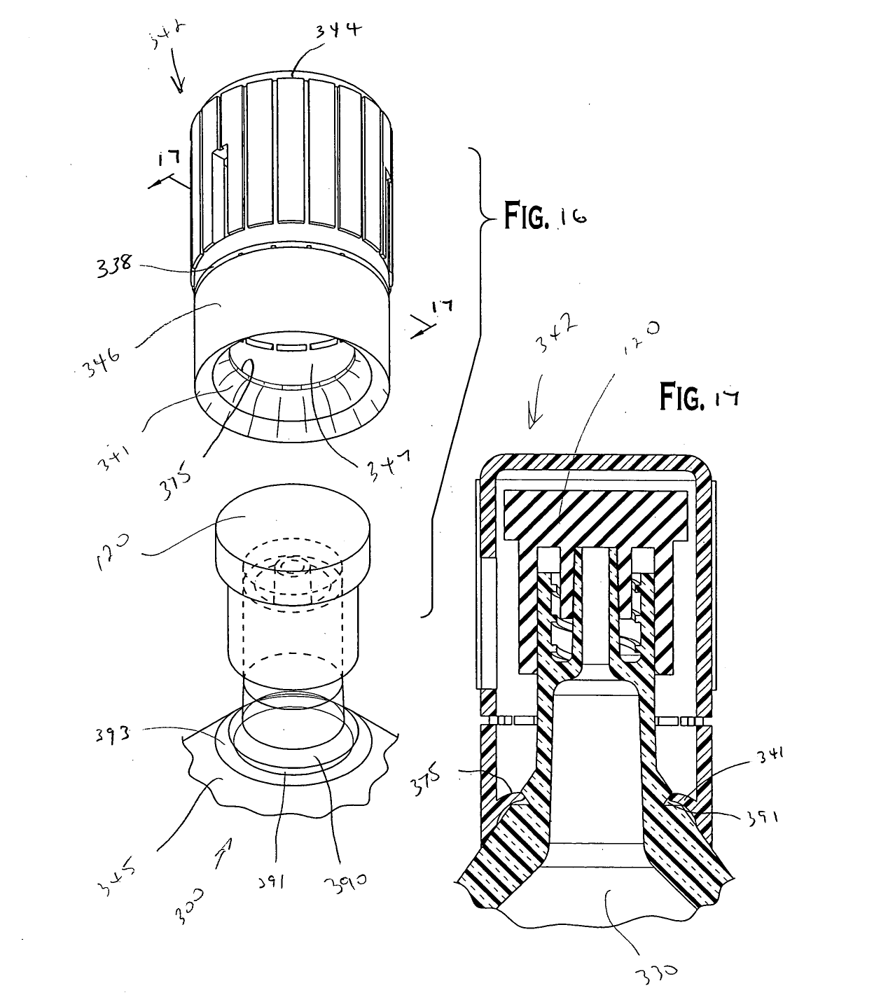

FIG. 4 shows a top plan view of the overcap 24 having top portion 34 and bottom portion 36.

FIG. 5 shows a bottom plan view of the overcap 24 having cut-outs or notches 40 and 40′ on opposite sides of the bottom portion 36. The bottom portion further comprises a rim or flange 41 extending inward from the proximal end 37, except it lacks continuity at cut-outs or notches 40 and 40′. The height of rim 41 is larger than the height of the ring 35 on the barrel so that the overcap 24 cannot be removed from the barrel without other manipulations described later in the process of using the overcap in preventing unauthorized use of the content of the barrel.

FIG. 6 shows a side-elevational view of the overcap 24 comprising: top portion 34, bottom portion 36 which are separated by a frangible portion 38. The proximal end 37 is provided with two cut-outs or notches 40 and 40′ on opposite sides of the bottom portion. The notches are designed to engage protuberances or knobs 26 and 26′ at the distal end of the barrel.

FIGS. 7, 8, 9 and 10 show another embodiment of the overcap, generally designated at 42, in various views. Unlike the previously described overcap wherein the bottom portion has a slightly larger diameter as compared to the diameter of the top portion, in this embodiment the diameters of the bottom and top portions of the overcap are the same.

FIG. 7 is a perspective view of the overcap comprising: a top portion 44, and a bottom portion 46 separated by a frangible portion 48. The proximal end 50 of the bottom portion is provided with two cut-outs or notches 52 and 52′ on the opposite sides of the bottom portion. The notches are designed to engage protuberances or knobs 26 and 26′ at the distal end of the barrel.

FIG. 8 shows a top plan view of the overcap 42 in which top portion 44 and bottom portion 46 have the same diameter.

FIG. 9 shows a bottom plan view of the overcap 42 having cut-outs or notches 52 and 52′ on opposite sides of the bottom portion 46. The bottom portion further comprises a rim or flange 54 extending inward from the proximal end 50, except it lacks continuity at cut-outs or notches 52 and 52′. The height of rim or flange 54 is larger than the height of the ring 35 on the barrel so that the overcap 42 cannot be removed from the barrel without other manipulations described later in the process of using the overcap in preventing unauthorized use of the content of the barrel.

FIG. 10 shows a side-elevational view of the overcap 42 comprising: top portion 44 and bottom portion 46 which are separated by a frangible portion 46. The diameters of the top and bottom portions are equal. The proximal end 50 is provided with two cut-outs or notches 52 and 52′ on opposite sides of the bottom portion. The notches are designed to engage protuberances or knobs 26 and 26′ at the distal end of the barrel.

The overcaps 24 and 42, respectively in the first two embodiments of the present invention, may be made of a polymeric material including but not limited to: polyolefins such as polyethylene and polypropylene; polystyrene, polycarbonate, polymethylpentene, cyclic olefin co-polymers, acrylic polymers and methacrylic polymers.

When the overcap is positioned over the distal end of the barrel, it may be turned clockwise or counter-clockwise without resulting in the removal of the overcap.

Personnel not familiar with the proper removal procedure will not be able to remove the overcap. The proper removal of the overcap is as follows: the overcap is pressed down towards the barrel until the cut-outs or notches 40 and 40′ or 52 and 52′, respectively, engage protuberances or knobs 26 and 26′ on the distal end of the barrel.

Upon such engagement the overcap will not turn either clockwise or counter-clockwise direction. The top portion of the overcap 34 and 44 respectively, is then turned clockwise or counter-clockwise to separate the top portions from the bottom portions 36 and 46, respectively. The frangible portions 38 and 48, respectively, allow such separation of the top and bottom portions. While the bottom portions remain on the distal end of the barrel, the top portions are removed to expose the resilient closures 20 upon the removal of which the female luer connector 28 is exposed. To the female luer connector a male luer connector, having an injection needle or IV tubing, is attached for withdrawal of the content of the barrel.

The following are particular embodiments regarding the tamper evident cap and prefilled syringe combinations of the invention:

1. A combination of a tamper evident protective cap and a prefilled glass or plastic syringe or cartridge barrel or a tube stoppered by an elastomeric closure at its tapered distal end wherein said syringe or cartridge barrel or tube.

The syringe or cartridge barrel or tube comprises a cylindrical chamber having a tapered distal end terminating in a tip having a bore therethrough, said bore is stoppered by an elastomeric closure, said cylindrical chamber containing a liquid therein, wherein said tip is equipped with a female luer connector to which a male luer connector or an injection needle may be attached, a pair of protuberances at the tapered distal end on the opposite sides of the barrel designed to receive and engage a pair of notches on the proximal end of a tamper evident protective cap, a ring on the tapered distal end of the barrel spaced between said pair of protuberances and said female luer connector.

The tamper evident protective cap is removably engaged with said tapered distal end of said syringe or cartridge barrel or tube and comprises a cylindrical top portion and a cylindrical bottom portion connected by a frangible area allowing the top portion to be removed from the bottom portion.

The cylindrical top portion terminates in a flat circular surface conforming to said elastomeric closure in said tip of the syringe or cartridge barrel or tube; said bottom portion terminates in an open end comprising a flange extending inwardly designed to prevent removal of the tamper evident protective cap; and a pair of notches on the proximal end of said tamper evident protective cap engaging said pair of protuberances on the tapered distal end of the syringe or cartridge barrel or tube.

Upon engagement of said pair of notches with said pair of protuberances on the top portion of said tamper evident protecting cap is twistably separated from the bottom portion thereof thereby exposing the tapered tip along with the elastomeric closure and female luer connector ready for withdrawal of said liquid from said syringe or cartridge barrel or tube.

2. The combination of a tamper evident protective cap and a prefilled glass or plastic syringe or cartridge barrel or a tube of embodiment 1 in which said cylindrical bottom portion is slightly larger than said cylindrical top portion and separated by said frangible portion therebetween.

3. The combination of a tamper evident protective cap and a prefilled glass or plastic syringe or cartridge barrel or a tube of embodiment 1 in which said cylindrical bottom portion and said cylindrical top portion are of the same size separated by said frangible portion therebetween.

4. The combination of a tamper evident protective cap and a prefilled glass or plastic syringe or cartridge barrel or a tube of embodiment 1 in which said ring on the tapered distal end of the barrel prevents removal of said tamper evident protective cap prior to separation of said bottom and top portions of said tamper evident protective cap.

5. The combination of a tamper evident protective cap and a prefilled glass or plastic syringe or cartridge barrel or a tube of embodiment 1 in which said tamper evident protective cap is made of a polymer material selected from the group consisting of polyethylene, polypropylene, polystyrene, polycarbonate, polymethylpentene, cyclic olefin co-polymers, acrylic polymers and methacrylic polymers.

6. The combination of a tamper evident protective cap and a prefilled glass or plastic syringe or cartridge barrel or a tube of embodiment 1 in which said liquid in said syringe or cartridge barrel or tube is a pharmaceutical or biological liquid.

7. The combination of a tamper evident protective cap and a syringe or cartridge barrel or a tube of embodiment 6 in which said pharmaceutical or biological liquid is sterilized.

8. The combination of a tamper evident protective cap and a prefilled syringe or cartridge barrel or a tube of embodiment 7 in which said sterilization is by autoclave.

9. The combination of a tamper evident protective cap and a prefilled syringe or cartridge barrel or a tube of embodiment 1 in which said elastomeric closure is a soft rubber stopper.

10. A method of delivering a pharmaceutical or biological liquid to a patient from a prefilled glass or plastic syringe or cartridge barrel or tube equipped with a tamper evident protective cap that provides a combination of a tamper evident protective cap and a prefilled syringe or cartridge barrel or tube.

The delivery comprises the steps of providing a glass or plastic syringe or cartridge barrel or tube containing a pharmaceutical or biological liquid therein, said syringe or cartridge barrel or tube comprising a cylindrical chamber having a tapered distal end terminating in a tip having a bore therethrough, said bore is stoppered by an elastomeric closure, wherein said tip is equipped with a female luer connector to which a male luer connector or an injection needle is attached, a pair of protuberances at the tapered distal end on the opposite sides of the barrel designed to receive and engage a pair of notches on the proximal end of a tamper evident protective cap, a ring on the tapered distal end of the barrel spaced between said pair of protuberances and said female luer connector.

The tamper evident protective cap is removably engaged with said tapered distal end of said syringe or cartridge barrel or tube and comprises a cylindrical top portion and a cylindrical bottom portion connected by a frangible area allowing the top portion to be removed from the bottom portion.

The cylindrical top portion terminates in a flat circular surface conforming to said elastomeric closure in said tip of the syringe or cartridge barrel or tube; said bottom portion terminates in an open end comprising a flange extending inwardly designed to prevent removal of the tamper evident protective cap; and a pair of notches on the proximal end of said tamper evident protective cap engaging said pair of protuberances on the tapered distal end of the syringe or cartridge barrel or tube.

Upon engagement of said pair of notches with said pair of protuberances on the top portion of said tamper evident protecting cap is twistably separated from the bottom portion thereof thereby exposing the tapered tip along with the elastomeric closure and female luer connector ready for withdrawal of said liquid from said syringe or cartridge barrel or tube.

The steps further include engaging said pair of notches with said pair of protuberances on the top portion of said tamper evident protective cap, separating said top portion from said bottom portion of the protecting cap by twisting thereby exposing the tapered tip along with the elastomeric closure and female luer connector, removing the elastomeric closure from the tip of the syringe or cartridge barrel or tube, connecting a male luer connector or a syringe equipped with a male luer connector to the female luer connector and delivering the pharmaceutical or biological liquid to the patient by advancing a plunger in said syringe or cartridge barrel or tube towards the distal end thereof.

11. The method of embodiment 10 in which said pharmaceutical or biological liquid is an x-ray contrast medium.

The disclosure will now be directed to the embodiments that are illustrated and shown in FIGS. 11-19.

FIGS. 11-12 show an elevated perspective view and a cross-sectional view taken along line 12-12 of FIG. 11 of a fuirther embodiment of a tamper evident protective cap and prefilled syringe combination. In this embodiment, the combination includes a tamper evident overcap 142, a prefilled syringe 100 and a resilient or elastomeric closure 120.

FIG. 13 shows a perspective view of the prefilled syringe 100. Prefilled syringe 100 includes a syringe barrel 105 of cylindrical configuration. Syringe barrel 105 has a longitudinal axis A that extends between a distal end 140 and a proximal end 160. Syringe barrel 105 also has an inner surface 112 that defines a cylindrical chamber 130. The cylindrical chamber 130 houses a medical fluid 110 that is disposed therein. Syringe barrel 105 further provides a tapered tip 150 that terminates at the distal end 140. The tapered tip 150 has a bore 155 that extends therethrough. The bore 155 is stoppered by the elastomeric closure 120.

The prefilled syringe 100 may optionally include a plunger 180 that is received through the proximal end 160 and is positioned within the syringe barrel 105. Plunger 180 is adapted for movement along the longitudinal axis. The materials used to make the plunger 180 are conventional and known to those skilled in the art.

In a preferred embodiment, the plunger may be made of one, two or more pieces. The plunger may, for example, be a single piece component, or a two piece component consisting of a core and a flexible cover piece attached to or fitting over onto the core (e.g., allowing the plunger to seal the barrel of the syringe). In the latter case, the core is preferably made of a relatively hard plastic such as a polyolefin (e.g., polypropylene or polycarbonate), and the flexible rubber piece is preferably made of a flexible rubber elastomer, such as natural rubber, butyl or halobutyl rubber; the two pieces may be pre-assembled to form the plunger prior to insertion into the barrel.

The tapered tip 150 is further equipped with a Luer lock collar 170. The tapered tip 150 and Luer lock collar 170 are designed International Standard (ISO 594-2) entitled, “Conical fittings with 6% (Luer) taper for syringes, needles and certain other medical equipment”. The luer collar 170 may optionally provide a threaded connection 132 to which an injection needle or a luer connector equipped with a tubing conduit (not shown) can be attached.

The syringe barrel 105 may be made of any suitable plastic, and is preferably made of polyolefin, including polyolefin polymers, polyolefin copolymers and blends, especially polypropylene or blends thereof with polyethylene, or olefin polymers and copolymers, including methylpentene or the like polyolefins, and cyclic olefins or polyester.

The elastomeric closure 120 may be made of any suitable plastic or combination of plastic and elastomer, and is preferably made of a flexible rubber elastomer such as natural rubber, butyl or halobutyl rubber or blends thereof.

Syringe barrel 105 may optionally also include a flat ring 119 that extends away from the longitudinal axis and is disposed at the proximal end 160 of the syringe barrel 105. Flat ring 119 serves as a mechanical locating device for positioning the syringe assembly at a filling station (not shown). In addition, flat ring 119 may serve to prevent the medical fluid from flowing into the injection device during use. Flat ring 119 may be made from any suitable plastic, and is preferably made from the same family of materials that were disclosed above in connection with the syringe barrel 105.

The syringe barrel 105 additionally includes a ring 190 that is disposed at a distal end 145 of the cylindrical chamber 130. The tamper evident cap 142 is removably engaged with the ring 190 that is disposed at the distal end 145 of the cylindrical chamber 130. The tamper evident cap 142 includes a cylindrical top portion 144 that terminates in a flat circular surface 145 and conforms to the elastomeric closure 120.

The tamper evident cap 142 further includes a cylindrical bottom portion 146 that is connected to the cylindrical top portion 144 by a frangible area 138. The frangible area or seal 138 allows the cylindrical top portion 144 to be removed from the cylindrical bottom portion 146. The cylindrical bottom portion 146 terminates in an open end 147 and has a flange or rim 141 that extends inwardly. An upper portion 175 of the flange 141 is designed to engage and lock into a lower portion 195 of the ring 190.

Once the flange 141 is locked into the ring 190, the top portion 144 of tamper evident protective cap 142 can not be removed from the bottom portion 146 without first breaking the frangible area or seal 138. If the frangible seal 138 is broken, the bottom portion 146 will remain engaged to the ring 190 and the disengaged top portion 144 will indicate tamper evidence to the medical practitioner by virtue of being disconnected from the combination.

In addition to the lower portion 195, the ring 190 may include an upper flat portion 177, a tapered portion 179, a side portion 181 and an undercut portion 183. The ring 190 may be molded as a single component of the syringe barrel 105 or may be fabricated separately and bonded to the syringe barrel. It will be recognized by those skilled in the art that there are numerous ways to bond one plastic to another. Those methods include, but are not limited to, heat welding, sonic welding and the use of adhesives or epoxies.

The cylindrical top portion 144 of the tamper evident protective cap 142 may optionally provide at least one air vent 149. The at least one air vent allows moisture to be driven out of the distal end 140 of the prefilled syringe 100 during a steam sterilization process. In another embodiment, the cylindrical top portion 144 may optionally provide three air vents 149 that allow moisture to be driven out of the distal end 140 of the syringe 100 during a steam sterilization process. It will be recognized by those skilled in the art that bacteria thrive and grow in wet and or moist environments. Therefore, if moisture is left in the syringe after a steam sterilization process there is a reasonable expectation that bacteria will begin to grow inside the syringe. This will result in loss of the product.

The cylindrical top portion 144 may also include a plurality of ribs 148 that are disposed on an outer surface 153 of the cylindrical top portion 144. The plurality of ribs 148 provides a medical practitioner with an improved grip for grasping the cylindrical top portion 144. In another embodiment, the cylindrical top portion 144 may provide at least twelve ribs 148 that are disposed on the outer surface 153 of the cylindrical top portion 144, also for the purpose of improving the grip of a medical practitioner.

In one embodiment, the cylindrical top portion 144 has a first diameter and the cylindrical bottom portion 146 has a second diameter. In this embodiment, the first diameter is larger than the second diameter. In a further embodiment, the first diameter is about 0.3 mm to 1.3 mm larger than the second diameter. In yet a further embodiment, the first diameter is about 0.8 mm larger than the second diameter.

In another embodiment, the syringe 100 may further comprise a means of releasably engaging an injection device. Such means are known to those skilled in the art and may include, for example, a locking mechanism disposed on the syringe barrel. For example, in the case of a front loading device, syringe barrel 104 may optionally include at least one locking tab that is disposed at the proximal end 105 of the syringe barrel 104 for interfacing with an injection device. The injection device may be hand held, semi-automatic or automatic.

In another embodiment, the at least one locking tab may comprise a first pair of locking tabs 107 that are positioned about 180° opposite a second pair of locking tabs 109. Both the first pair and second pair of locking tabs 107, 109 are disposed on an outer surface of the syringe barrel 104 and about the longitudinal axis. This embodiment may optionally further include a third locking tab 111 that is positioned about 180° opposite a fourth locking tab 113. Both the third locking tab 111 and fourth locking tab 113 are disposed on the outer surface of the syringe barrel 104 and about the longitudinal axis. The first and second pairs of locking tabs 107, 109 are 90° offset from the third and fourth locking tabs 111, 113.

A method of removing the tamper evident cap 142 from the pre-filled syringe 100 will now be described. The method includes the steps of 1) providing the tamper evident cap 142 having cylindrical top portion 144 and cylindrical bottom portion 146. The cylindrical top portion 144 being connected to cylindrical bottom portion 146 at frangible area 138, 2) holding the cylindrical bottom portion 146 with a first hand (not shown) and 3) grasping and twistably separating the cylindrical top portion 144 with a second hand (not shown), the top portion 144 separating from the bottom portion 146 at the frangible area 138.

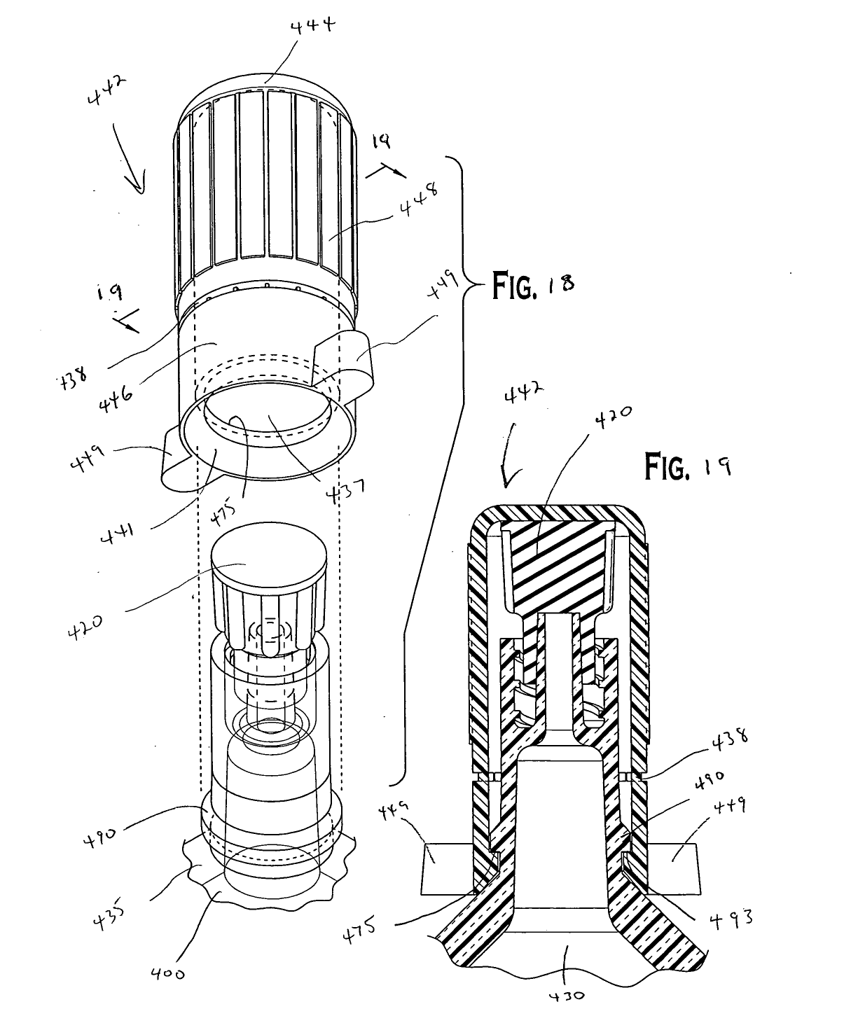

FIGS. 14-15 show an elevated perspective view and a cross-sectional view taken along line 15-15 of FIG. 14 of a further embodiment of a tamper evident protective cap and prefilled syringe combination. The combination includes tamper evident overcap 142, a prefilled syringe 200 and elastomeric closure 120.

In this embodiment, all of the features of the tamper evident cap and prefilled syringe combination of the present invention as described above with respect to FIGS. 11-13 are the same except for the following. Instead of providing the ring 190 that is disposed at the distal end 145 of the cylindrical chamber 130, at least two tabs 290 that project away from the longitudinal axis are provided. The at least two tabs 290 are disposed at a distal end 245 of cylindrical chamber 230.

The upper portion 175 of the flange 141 is designed to engage and lock into undercut portions 295 of the at least two tabs 290. Once the flange 141 is locked into the undercut portions 295, the top portion 144 of tamper evident protective cap 142 can not be removed from the bottom portion 146 without first breaking the frangible area or seal 138. If the frangible seal 138 is broken, the bottom portion 146 will remain engaged to the at least two tabs 290 and the disengaged top portion 144 will indicate tamper evidence to the medical practitioner by virtue of being disconnected from the combination.

FIGS. 16-17 show an elevated perspective view and a cross-sectional view taken along line 17-17 of FIG. 16 of a further embodiment of a tamper evident protective cap and prefilled syringe combination. The combination includes a tamper evident overcap 342, a prefilled syringe 300 and elastomeric closure 120.

In this embodiment, all of the features of the tamper evident cap and prefilled syringe combination of the present invention as described above with respect to FIGS. 11-13 are the same except for the following. Instead of providing the ring 190 that is disposed at the distal end 145 of the cylindrical chamber 130, a shoulder 390 that is disposed at a distal end 345 of cylindrical chamber 330 is provided. The shoulder 390 has an annular groove 391. The annular groove 391 is disposed on an outer surface 393 of the shoulder 390.

The tamper evident cap 342 further includes a cylindrical bottom portion 346 that is connected to the cylindrical top portion 344 by a frangible area 338. The frangible area 338 allows the cylindrical top portion 344 to be removed from the cylindrical bottom portion 346. The cylindrical bottom portion 346 terminates in an open end 347 and has a flange 341 that extends inwardly. An upper portion 375 of the flange 341 is designed to engage and lock into the grove 391 of the shoulder 390.

In one embodiment, the flange 341 may be biased toward the distal end 345. In another embodiment, the flange 341 may be biased away from the distal end 345. In both cases, the flange 341 locks and engages the groove 391 such that the tamper evident cap 342 can not be removed without tamper evidence.

In one embodiment, the annular groove 391 may be provided v-shaped. In another embodiment, the annular groove 391 may be provided semi-circular.

It will be recognized by those skilled in the art that there are an unlimited number of different geometrical configurations that the annular groove and flange could be configured to and that the scope of the invention should not be limited only to the two embodiments disclosed.

Once the flange 341 is locked into the groove 391, the top portion 344 of tamper evident protective cap 342 can not be removed from the bottom portion 346 without first breaking the frangible area or seal 338. If the frangible seal 338 is broken, the bottom portion 346 will remain engaged to the annular groove 391 and the disengaged top portion 344 will indicate tamper evidence to the medical practitioner by virtue of being disconnected from the combination.

FIGS. 18-19 show an elevated perspective view and a cross-sectional view taken along line 19-19 of FIG. 18 of a further embodiment of a tamper evident protective cap and prefilled syringe combination. The combination includes a tamper evident overcap 442, a prefilled syringe 400 and an elastomeric closure 420.

In this embodiment, all of the features of the tamper evident cap and prefilled syringe combination of the present invention as described above with respect to FIGS. 11-13 are the same except for the following. Instead of providing tamper evident cap 142, tamper evident cap 442 is provided. In addition, elastomeric closure 420 is provided instead of elastomeric closure 120. In this embodiment, elastomeric closure 420 only covers a portion of the tapered tip. Whereas, in the previous embodiments, elastomeric closure 142 covers the tapered tip and Luer lock collar.

Tamper evident cap 442 includes a cylindrical top portion 444 and a cylindrical bottom portion 446. The cylindrical top portion 444 is connected to the cylindrical bottom portion 446 at frangible area 438. The cylindrical bottom portion 446 has an open end 437 and a flange 441 that projects inward toward the open end. The cylindrical bottom portion 446 additionally includes at least two wings 449 that project away from the longitudinal axis. The at least two wings 449 provide an additional surface for the medical practitioner to grip when attempting to separate the cylindrical top portion 444 from the cylindrical bottom portion 446 at the frangible seal 438.

An upper portion 475 of the flange 441 is designed to engage and lock into a lower portion 493 of the ring 490. Once the flange 441 is locked into the lower portion 493 of the ring 490, the top portion 444 of tamper evident protective cap 442 can not be removed from the bottom portion 446 without first breaking the frangible area or seal 438. If the frangible seal 438 is broken, the bottom portion 446 will remain engaged to the ring 490 and the disengaged top portion 444 will indicate tamper evidence to the medical practitioner by virtue of being disconnected from the combination.

With respect to the all of the disclosed tamper evident overcap and prefilled syringe embodiments, when the top portions of the overcaps are separated from the bottom portions along the frangible areas the two portions cannot be reconnected without notice by the healthcare practitioner. If the overcap is tampered with, the healthcare professional will readily observe the separated top and bottom portions of the overcap along the frangible portion therebetween. As a result of this tamper evidence the content of the barrel will not be used.

Additionally, all of the embodiments of the present invention are used with prefilled barrels containing, for example, a biological liquid or a pharmaceutical such as contrast media. Subsequent to the prefill the tamper evident overcap is positioned on the distal end of the barrel, such as by crimping or pressing the overcap onto the distal end of the barrel.

1. A combination of a tamper evident protective cap and a pre-filled syringe, the combination comprising:
a) a syringe barrel of cylindrical configuration having a longitudinal axis extending between a distal end and a proximal end, the syringe barrel including:
1) an inner surface defining a cylindrical chamber having a distal end;
2) a tapered tip terminating at the distal end of the syringe barrel, the tip having a bore therethrough, the bore stoppered by an elastomeric closure, the tip equipped with a Luer lock collar; and
3) a ring disposed at the distal end of the cylindrical chamber; and
b) a tamper evident cap removably engaged with the distal end of the syringe barrel and comprising:
1) a cylindrical top portion terminating in a flat circular surface conforming to the elastomeric closure disposed in the tapered tip of the syringe barrel; and
2) a cylindrical bottom portion connected to the cylindrical top portion by a frangible area, the frangible area allowing the cylindrical top portion to be removed from the cylindrical bottom portion, the cylindrical bottom portion terminating in an open end and comprising a flange extending inwardly designed to engage the ring such that the engagement prevents removal of the tamper evident protective cap unless the top portion is intentionally separated from the bottom portion at the frangible area, the bottom portion remaining engaged to the ring, the disengaged top portion indicating tamper evidence.

2. The combination of claim 1 wherein the cylindrical top portion of the tamper evident protective cap further comprises at least one air vent.

3. The combination of claim 1 wherein the cylindrical top portion further comprises a plurality of ribs.

4. The combination of claim 1 wherein the cylindrical top portion further comprises three air vents.

5. The combination of claim 1 wherein the cylindrical top portion further comprises twelve ribs.

6. The combination of claim 1 wherein the cylindrical top portion comprises a first diameter and the cylindrical bottom portion comprises a second diameter and wherein the first diameter is larger than the second diameter.

7. The combination of claim 1 wherein the cylindrical top portion comprises a first diameter and the cylindrical bottom portion comprises a second diameter and wherein the first diameter is about 0.3 mm to 1.3 mm larger than the second diameter.

8. The combination of claim 1 wherein the cylindrical top portion comprises a first diameter and the cylindrical bottom portion comprises a second diameter and wherein the first diameter is about 0.8 mm larger than the second diameter.

9. The combination of claim 1 wherein the syringe barrel comprises a material selected from the group consisting of polyolefin polymers, polyolefin copolymers, polypropylene, olefin polymers, olefin copolymers, cyclic olefins, polyester or methylpentene.

10. The combination of claim 1 wherein said tamper evident protective cap comprises a polymer material selected from the group consisting of polyethylene, polypropylene, polystyrene, polycarbonate, polymethylpentene, cyclic olefin co-polymers, acrylic polymers and methacrylic polymers.

11. The combination of claim 1 wherein the elastomeric closure comprises a material selected from the group consisting of natural rubber, butyl or halobutyl rubber.

12. The combination of claim 1 wherein the elastomeric closure comprises a plastic, an elastomeric compound or a combination of plastic and elastomeric compound.

13. A method of removing a tamper evident cap from a pre-filled syringe comprising the steps of:
a) providing a tamper evident cap comprising:
1) a cylindrical top portion; and
2) a cylindrical bottom portion connected to the cylindrical top portion at a frangible seal; and
b) holding the cylindrical bottom portion with a first hand; and
c) grasping and twistably separating the cylindrical top portion with a second hand, the top portion separating from the bottom portion at the frangible seal.

14. The combination of claim 1 further comprising a means of releasably engaging an injection device;

15. The combination of claim 1 further comprising a locking mechanism disposed on the syringe barrel for interfacing with an injection device;

16. The combination of claim 1 further comprising at least one locking tab disposed at the proximal end of the syringe barrel for interfacing with an injection device;

17. The combination of claim 16 wherein the at least one locking tab further comprises:
(a) a first pair of locking tabs positioned 180° opposite a second pair of locking tabs disposed on an outer surface of the syringe barrel and about the longitudinal axis; and
(b) a third locking tab positioned 180° opposite a fourth locking tab disposed on the outer surface of the syringe barrel and about the longitudinal axis, wherein the first and second pairs of locking tabs are 90° offset from the third and fourth locking tabs.

18. The combination of claim 17 further comprising a flat ring extending away from the longitudinal axis and disposed at the proximal end of the syringe barrel.

19. A combination of a tamper evident protective cap and a pre-filled syringe, the combination comprising:
a) a syringe barrel of cylindrical configuration having a longitudinal axis extending between a distal end and a proximal end, the syringe barrel including:
1) an inner surface defining a cylindrical chamber having a distal end; and
2) a tapered tip terminating at the distal end, the tip having a bore therethrough, the bore stoppered by an elastomeric closure, the tip equipped with a Luer lock collar; and
3) at least two tabs projecting outward and disposed at the distal end of the cylindrical chamber; and
b) a tamper evident cap removably engaged with the tapered distal end of the syringe barrel and comprising:
1) a cylindrical top portion terminating in a flat circular surface conforming to the elastomeric closure disposed in the tapered tip of the syringe barrel; and
2) a cylindrical bottom portion connected to the cylindrical top portion by a frangible area, the frangible area allowing the cylindrical top portion to be removed from the cylindrical bottom portion, the cylindrical bottom portion terminating in an open end and comprising a flange extending inwardly designed to engage the at least two tabs such that the engagement prevents the removal of the tamper evident protective cap unless the top portion is intentionally separated from the bottom portion at the frangible area, the bottom portion remaining engaged to the at least two tabs, the disengaged top portion indicating tamper evidence.

20. A combination of a tamper evident protective cap and a pre-filled syringe, the combination comprising:
a) a syringe barrel of cylindrical configuration having a longitudinal axis extending between a distal end and a proximal end, the syringe barrel including:
1) an inner surface defining a cylindrical chamber having a distal end; and
2) a tapered tip terminating at the distal end, the tip having a bore therethrough, the bore stoppered by an elastomeric closure, the tip equipped with a Luer lock collar; and
3) a shoulder disposed at the distal end of the cylindrical chamber, the shoulder having an annular groove disposed on an outer surface; and
b) a tamper evident cap removably engaged with the distal end of the syringe barrel and comprising:
1) a cylindrical top portion terminating in a flat circular surface conforming to the elastomeric closure disposed in the tapered tip of the syringe barrel; and
2) a cylindrical bottom portion connected to the cylindrical top portion by a frangible area, the frangible area allowing the cylindrical top portion to be removed from the cylindrical bottom portion, the cylindrical bottom portion terminating in an open end and comprising a flange extending inwardly designed to engage the groove such that the engagement prevents the removal of the tamper evident protective cap unless the top portion is intentionally separated from the bottom portion at the frangible area, the bottom portion remaining engaged to the annular groove, the disengaged top portion indicating tamper evidence.

21. The combination of claim 20 wherein the annular groove is v-shaped.

22. The combination of claim 20 wherein the annular groove is semi-circular in shape.

23. The combination of claim 20 wherein the flange is biased away from the distal end.

24. The combination of claim 20 wherein the flange is biased toward the distal end.

25. A combination of a tamper evident protective cap and a pre-filled syringe, the combination comprising:
a) a syringe barrel of cylindrical configuration having a longitudinal axis extending between a distal end and a proximal end, the syringe barrel including:
1) an inner surface defining a cylindrical chamber having a distal end; and
2) a tapered tip terminating at the distal end, the tip having a bore therethrough, the bore stoppered by an elastomeric closure, the tip equipped with a Luer lock collar; and
3) a ring disposed at the distal end of the cylindrical chamber; and
b) a tamper evident cap removably engaged with the tapered distal end of the syringe barrel and comprising:
1) a cylindrical top portion terminating in a flat circular surface conforming to the elastomeric closure disposed in the tapered tip of the syringe barrel; and
2) a cylindrical bottom portion connected to the cylindrical top portion by a frangible area, the frangible area allowing the top portion to be removed from the bottom portion, the bottom portion terminating in an open end and comprising:
a) at least two wings projecting away from the longitudinal axis; and
b) a flange extending inwardly designed to engage the ring such that the engagement prevents the removal of the tamper evident protective cap unless the top portion is intentionally separated from the bottom portion at the frangible area, the bottom portion remaining engaged to the ring, the disengaged top portion indicating tamper evidence.
